(19) United States
(12) Patent Application Publication
(10) Pub. No.: US 2023/0135500 A1
(43) Pub. Date: May 4, 2023

(54) NASAL SWAB

(71) Applicant: CITY OF HOPE, Duarte, CA (US)

(72) Inventors: Yuman Fong, La Canada, CA (US); Colin Andrew Cook, Monrovia, CA (US); Thomas J Gernon, Sierra Madre, CA (US); Stanley Ralph Hamilton, Altadena, CA (US); Marylin Roque, Los Angeles, CA (US); Kevin Alexander Proudfoot, San Francisco, CA (US); Sophie Yu, Pasadena, CA (US); Natalie Krieger, Gilbert, AZ (US); Vivian Ho Lam, Garden Grove, CA (US); Kaitlyn Paulsen, Seattle, WA (US); Kaushal Gokli, Upland, CA (US); Leila Wiberg, Claremont, CA (US); Tristan Aniken Huang, Claremont, CA (US); Vadim Peter Mathys, Boulder, CO (US)

(21) Appl. No.: 17/976,670

(22) Filed: Oct. 28, 2022

Related U.S. Application Data
(60) Provisional application No. 63/275,293, filed on Nov. 3, 2021.

(51) Int. Cl. A61B 10/02; A61B 10/00

(52) U.S. Cl. CPC A61B 10/02; A61B 10/0045

(57) ABSTRACT

Methods of manufacture, devices, and systems are described for a nasal swab. The swab includes an elongated body having a distal end. The swab includes a collection layer proximate to the distal end of the elongated body. The collection layer has a first geometric layout and configured to collect samples in the nasal passageway. The swab includes a collection tip at the distal end of the elongated body, the collection tip having a second geometric layout and configured to collect samples in the nasal passageway. The first geometric layout is different from the second geometric layout.

### TECHNICAL FIELD

The present disclosure relates generally to medical devices, and more particularly, to a nasal swab.

### BACKGROUND

Nasal swabs collect samples to detect pathogens. For example, nasal swabs may collect nasal liquid samples for detecting COVID-19. But nasal swabs cause patient discomfort due to the depth at which the nasal swab must be inserted into a patient's nose. Further, a traditional nasal swab tends to be less reliable because its small sample size may result in false negatives. These false negatives result in undiagnosed diseases and their potential spread. Current nasal swabs are also uncomfortable and do not collect a sufficiently large sample from the nasal passageway. This can be especially problematic for the detection of respiratory pathogens and the spread of disease, such as COVID-19.

### SUMMARY

The present disclosure provides methods, systems, and articles of manufacture for a nasal swab for collecting samples in a nasal passageway.

In one aspect, there is provided a swab including an elongated body having a distal end. The swab includes an elongated body having a distal end. The swab includes a collection layer proximate to the distal end of the elongated body. The collection layer has a first geometric layout and configured to collect samples in the nasal passageway. The swab includes a collection tip at the distal end of the elongated body, the collection tip having a second geometric layout and configured to collect samples in the nasal passageway. The first geometric layout is different from the second geometric layout.

In some variations, the collection layer is coupled to the collection tip at the distal end of the elongated body. Additionally, the first geometric layout of the collection layer forms an inner cavity configured to retain samples from the nasal passageway. Further, the inner cavity is situated below an outer surface formed by the first geometric layout of the collection layer. Additionally, the collection tip is configured to cover an opening of the inner cavity.

In some variations, the collection layer and the collection tip are made of silicone, and wherein cylindrical bristles of the collection layer and the collection tip have a stiffness matching a nasal tissue stiffness. Further, the cylindrical bristles have a triangular pattern, and wherein ends of the cylindrical bristles may have a hemispherical geometry to mitigate scraping against the nasal passageway to decrease patient discomfort. Additionally, the collection layer is formed around a circumference of the elongated body, and wherein the first geometric layout of the collection layer forms pores configured to retain samples from the nasal passageway. Additionally, the pores are configured to facilitate capillary action to retain samples from the nasal passageway. In some variations, the elongated body is rigid and the collection layer is flexible. Further, the first geometric layout includes at least one of bristles, spiked disks, interconnecting disks, and triangular disks, and wherein the second geometric layout is at least one of spirals, cylindrical protrusions, arches, and lateral-ringed arches. Additionally, the swab further comprises a proximate end of the elongated body, wherein the proximate end of the elongated body is configured to manipulate the swab for inserting the swab into the nasal passageway. Further, the elongated body is cylindrical and wherein the elongated body includes a hollow tube for transporting samples.

In another aspect, there is provided a swab system for collecting samples in a nasal passageway. The swab system includes an elongated body having a distal end. The swab system includes a collection layer proximate to the distal end of the elongated body. The collection layer has a first geometric layout and configured to collect samples in the nasal passageway. The swab system includes a collection tip at the distal end of the elongated body, the collection tip having a second geometric layout and configured to collect samples in the nasal passageway. The first geometric layout is different from the second geometric layout.

In some variations, the collection layer is configured to couple to the collection tip at the distal end of the elongated body. Further, the first geometric layout of the collection layer is configured to form an inner cavity configured to retain samples from the nasal passageway. Additionally, the collection tip is configured to cover an opening of the inner cavity.

In yet another aspect, there is provided a method of manufacturing a swab for collecting samples in a nasal passageway. The method includes forming a collection layer at a distal end of an elongated body, the collection layer having a first geometric layout and configured to collect samples in the nasal passageway. The method includes forming a collection tip at the distal end of the elongated body, the collection tip having a second geometric layout and configured to collect samples in the nasal passageway. The first geometric layout is different from the second geometric layout.

In some variations, the method further comprises applying a surface treatment to the collection layer and the collection tip, the surface treatment configured to positively charge the collection layer and the collection tip. Additionally, the method further comprises applying a coating to the collection layer and the collection tip, the coating configured to bond to pathogenic material located in the nasal passageway.

The details of one or more variations of the subject matter described herein are set forth in the accompanying drawings and the description below. Other features and advantages of the subject matter described herein will be apparent from the description and drawings, and from the claims. While certain features of the currently disclosed subject matter are described for illustrative purposes, it should be readily understood that such features are not intended to be limiting. The claims that follow this disclosure are intended to define the scope of the protected subject matter.

### BRIEF DESCRIPTION OF THE DRAWINGS

The embodiments herein may be better understood by referring to the following description in conjunction with the accompanying drawings in which like reference numerals indicate identically or functionally similar elements, of which:

FIG. 1 depicts an example of a swab including a collection layer and a collection tip;

FIG. 2 depicts an example of a model illustrating a collection layer having bristles;

FIG. 3 depicts another example of a model illustrating a collection layer having spiked disks;

FIG. 4 depicts another example of a model illustrating a collection layer having interconnecting disks;

FIG. 5 depicts another example of a model illustrating a collection layer having triangular disks;

FIG. 6 depicts an example of a model illustrating a collection tip having spirals;

FIG. 7 depicts another example of a model illustrating a collection tip having cylindrical protrusions;

FIG. 8 depicts another example of a model illustrating a collection tip having arches;

FIG. 9 depicts another example of a model illustrating a collection tip having lateral-ringed arches;

FIG. 10A depicts an example of a model illustrating a silicone collection layer and a silicone collection tip;

FIG. 10B depicts an example of a bristle stress test of a silicone cylindrical bristle;

FIG. 11 depicts an example of a fluid uptake test results between swabs having a flocked collection layer, a 3-D printed collection layer, and a silicone collection layer;

FIG. 12 depicts an example of display illustrating a swab being inserted into a nasal passageway; and

FIG. 13 depicts an example of a table illustrating manufacturing methods of a swab.

FIGS. 14A-F depict an example of a model illustrating a variety of colored and scented swabs.

FIGS. 15A-15C depict an example of a process of centrifugal elution of a sample from a swab.

### DETAILED DESCRIPTION

The nasal swab described herein increases the likelihood of accurate test results by collecting relatively larger samples compared to previous swabs. The nasal swab may be configured to collect larger samples with a collection layer and a collection tip. The large sample may also increase the likelihood of preserving and transporting viral nucleic acid (e.g., nucleic acid of COVID-19) and other pathogenic material for testing.

The swab may also decrease discomfort in patients. The depth at which the rigid tip of the nasal swab is inserted may cause discomfort. A conventional nasal swab has a rigid swab tip that may be required to travel 15-17 cm in length within the nasal passageway to collect samples. The nasal swab described here overcomes the problems of prior nasal swabs by having a flexible tip. Additionally, the flexible swab tip may be configured to optimize the contact area with the nasal passageway. Further, the swab may include a surface treatment to attract viral or genetic material. Additionally, abrasive layers near the tip may collect samples in both dry and wet environments. Additionally, the swab material may reduce the abrasiveness of the swab against the nasal passageway to reduce discomfort.

Previous swabs present problems during the elution process. The elution process extracts the sample collected by the swab for amplification and testing. With previous swabs, elution may only be 30% effective due to isolated sample pockets and difficulty removing the sample from the swab. But the nasal swab described herein corrects the problems of prior swabs by preventing isolated sample pockets and cleanly removing the sample from the swab. Additionally, the material selection of the swab described herein may increase the likelihood of a larger sample and reduce isolated sample pockets.

Additionally, the geometrical layout and arrangement of the disclosed abrasive layer and the collection tip may reduce the size of these pockets and may facilitate extraction of the sample from the swab. In some embodiments, a tube pipette may be integrated into the nasal swab to enhance the transportation of the sample. Additionally, the swab may be coated or have a surface treatment applied to enhance the collection of nasal fluid. As such, the swab may provide a superior sample size, comfort, and manufacturability compared to previous swabs for the detection of respiratory pathogens, including COVID-19.

The methods, systems, apparatuses, and manufacturing methods described herein provide a swab for collecting samples in a nasal passageway. The various exemplary embodiments also disclose a system for collecting samples in a nasal passageway as well as methods of manufacturing nasal swabs.

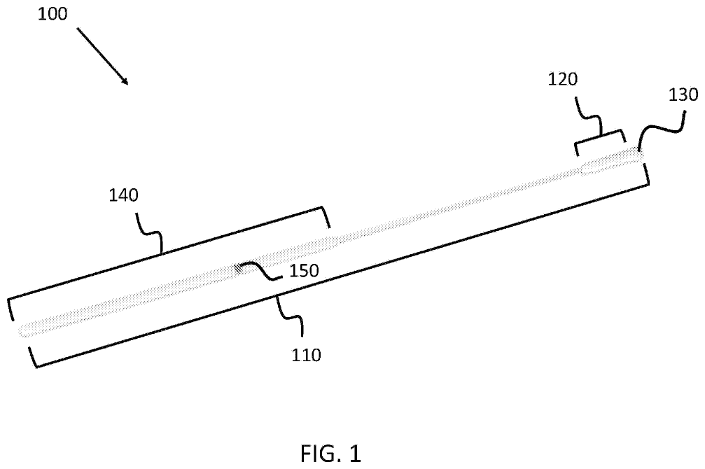

FIG. 1 depicts an example of a swab 100 including a collection layer 120 and a collection tip 130. The swab 100 may be configured to collect a large sample in comparison to previous swabs. The large sample may increase the likelihood of collecting and transporting viral nucleic acid and other pathogenic material for testing. The swab 100 may be configured to retain a sample from a nasal passageway, the nasopharynx, and/or the like. The swab 100 may include an elongated body 110 having a handle 140, a collection layer 120, and a collection tip 130. In some embodiments, the collection layer 120 and the collection tip 130 may be configured to couple to the elongated body 110.

The elongated body 110 may have a distal end and a proximate end. The collection layer 120 and the collection tip 130 may be located at the distal end of the elongated body 110. The handle 140 may be located at the proximate end of the elongated body 110. The elongated body 110 may be configured to be inserted into a nasal passageway. The elongated body 110 may be configured to be manipulated to insert the swab 100 into a nasal passageway. In some embodiments, the elongated body 110 may be rigid while the collection layer 120 and the collection tip 130 may be flexible. In some embodiments, the elongated body 110 may be cylindrical. In some embodiments, the elongated body 110 may be longer than 10 centimeters.

In some embodiments, the elongated body 110 may include a hollow tube for transporting samples. For example, the elongated body 110 may include a bulb pipette configured to pull in a sample from the nasal passageway and/or nasopharynx. Pulling in the sample with the bulb pipette enables the swab 100 to function as a nasal aspiration tube in addition to collecting samples. In some embodiments, the bulb pipette may extend from the handle 140 to the collection tip 130 for transporting the sample from the nasal passageway and/or nasopharynx. In some embodiments, the elongated body 110 may be made from acrylonitrile butadiene styrene, ethylene vinyl acetate, high-density polyethylene, thermoplastic polyurethane, low-density polyethylene, polyamide, polyether block amide, polyetheretherketone, polyethylene terephthalate, polycarbonate, polypropylene, polyvinyl alcohol, and/or polysiloxane.

The collection layer 120 may be proximate to the distal end of the elongated body 110 and can extend over a distal region of the elongated body 110. The collection layer 120 may be configured to couple to the distal end of the elongated body 110. The collection layer 120 may be formed around the circumference of the elongated body 110. The collection layer 120 may be configured to collect samples in the nasal passageway and/or nasopharynx. The collection layer 120 may include a geometric layout configured to enhance the sample collected from the nasal passageway and/or nasopharynx relative to previous swabs. The geometric layouts may include bristles 200, spiked disks 300, interconnecting disks 400, or triangular disks 500, which are non-limiting examples of the geometric layouts of the collection layer 120. In some embodiments, the collection layer 120 may be pre-wetted for improved specimen uptake. The collection layer 120 may be configured to rub, massage, or wipe the soft tissues of the nasal passageway and may have a low coefficient of friction to prevent irritation and abrasion of the soft tissues therein. The collection layer 120 may also have an atraumatic configuration so as to not to disrupt, irritate, or damage tissue when the collection layer contacts tissue.

The geometric layout of the collection layer 120 may form an inner cavity configured to retain a sample from the nasal passageway and/or nasopharynx. The inner cavity may be situated below an outer surface formed by the first geometric layout of the collection layer 120. The geometric layout of the collection layer 120 may form pores configured to retain samples from the nasal passageway and/or nasopharynx. The pores may be configured to facilitate capillary action to pull in and retain samples from the nasal passageway. The collection layer 120 may be made of flocked nylon, polyurethane foam, dacron, nylon, polyethylenes, polypropylene, polystyrene, acrylonitrile butadiene styrene, thermoplastic elastomers, thermoplastic polyurethane, silicone, and/or rayon.

The collection tip 130 may be situated at the distal end of the elongated body 110. The collection tip 130 may be configured to removably couple to the distal end of the elongated body 110. The collection tip 130 may be formed at the end of the elongated body 110 and may be formed to cover an opening at the collection layer 120. The collection tip 130 may be configured to collect samples in the nasal passageway and/or nasopharynx. The collection tip 130 may include a geometric layout configured to enhance the sample collected relative to previous swabs. The geometric layouts may include, for example, spirals 600, cylindrical protrusions 700, arches 800, and lateral-ringed arches 900. In some embodiments, the collection tip 130 is configured to cover an opening of the inner cavity. The collection tip 130 may be configured to rub, massage, or wipe the soft tissues of the nasal passageway and may have a low coefficient of friction to prevent irritation and abrasion of the soft tissues therein. The collection tip 130 may also have an atraumatic configuration so as to not to disrupt, irritate, or damage tissue when the collection layer contacts tissue.

The geometric layout of the collection tip 130 may form a tip cavity configured to retain a sample from the nasal passageway and/or nasopharynx. The inner cavity may be situated below an outer tip surface formed by the geometric layout of the collection tip 130. The geometric layout of the collection tip 130 may form pores configured to retain samples from the nasal passageway and/or nasopharynx. The pores may be configured to facilitate capillary action to retain samples from the nasal passageway. The collection tip 130 may be made of flocked nylon, polyurethane foam, dacron, nylon, polyethylenes, polypropylene, polystyrene, acrylonitrile butadiene styrene, thermoplastic elastomers, thermoplastic polyurethane, silicone, and/or rayon.

The handle 140 may be located at the proximate end or proximate region of the elongated body 110. The handle 140 may be configured to manipulate the swab 100 for inserting the swab 100 into the nasal passageway. In some embodiments, the handle 140 may include a hollow tube for transporting samples. For example, the handle 140 may include a bulb pipette configured to pull in the sample from the nasal passageway and/or nasopharynx. Pulling in the sample with the bulb pipette may enable the swab 100 to function as a nasal aspiration tube in addition to collecting samples. In some embodiments, the bulb pipette may extend from the handle 140 to the collection tip 130 for transporting the sample.

The handle 140 may include a pre-molded breakpoint 150 to lower the risk of sample contamination and to protect healthcare professionals from infection. The breakpoint 150 on the handle 140 may include an indent for an easy and clean break. The indent of the breakpoint 150 may allow healthcare professionals to snap the top half of the swab 100 that includes the collection layer 120 into a transport tube without needing to come into physical contact with the collection tip 130 and the sample material. The indent may have a smaller radius portion than the elongated body 110 and the indent may break when a stress threshold is satisfied.

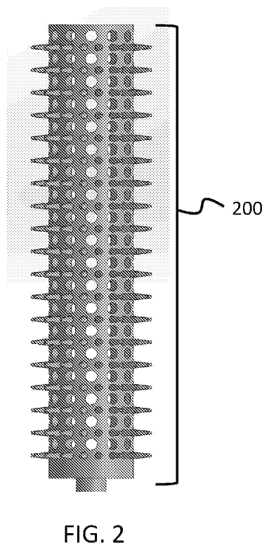

FIGS. 2-5 illustrate abrasive layers that may be situated at a distal region of the elongated body 110. The collection layer 120 may include a geometric layout configured to enhance the sample collected. FIGS. 2-5 illustrate various embodiments of a collection layer 120 having various geometric layouts that may include, for example, bristles 200, spiked disks 300, interconnecting disks 400, or triangular disks 500, which are non-limiting examples of the geometric layouts of the collection layer 120.

FIG. 2 depicts an example of a model illustrating a collection layer 120 having bristles 200. The collection layer 120 having bristles 200 may be configured to collect a large sample in comparison to previous swabs. The large sample may increase the likelihood of collecting and transporting viral nucleic acid and other pathogenic material for testing. The collection layer 120 having bristles 200 may also decrease discomfort in patients. The collection layer 120 with bristles 200 may be configured to retain a sample from a nasal passageway, the nasopharynx, and/or the like. The collection layer 120 with bristles 200 may facilitate elution capabilities, the performance in wet and dry nasal environments, and the decrease in the amount of material needed to manufacture the swab 100.

The collection layer 120 with bristles 200 may include bristles 200 extending from the elongated body 110. For example, the bristles 200 may radiate outward from the elongated body 110. The bristles 200 may extend outward along an axis perpendicular (or at any angle) to the axis of the elongated body 110. The bristles 200 may define an outer surface of the collection layer 120. The bristles 200 may be configured to capture sample material from the nasal passageway, the nasopharynx, and/or the like when positioned in contact with nasal passageway, the nasopharynx, and/or the like. The bristles 200 may form an inner cavity extending below the outer surface of the bristles 200. The inner cavity may be formed by an outer surface of the bristles 200. The bristles 200 may collect fluid using surface tension and abrade the contact area to collect the sample. The sample may become trapped between the bristles 200 and within the inner cavity formed by the outer surface of the bristles 200. In some embodiments, the collection layer 120 with bristles 200 may be configured to form pores. The pores may be configured to retain samples. The pores may be configured to facilitate a capillary action to capture and retain samples from the nasal passageway and/or the nasopharynx. In some embodiments, the bristles 200 may be tapered to enhance the porosity of the bristles 200. In some embodiments, the bristles 200 may have a hemispherical geometry at the bristle tip to prevent scraping against the nasal mucosa. For example, the bristles 200 may bend rather than scape when in contact with the nasal mucosa. The bristles 200 may bend well below the tensile strength of the nasal mucosa. For example, the bristles 200 may have the tensile strength of 150° per megapascal. In some embodiments, the bristles 200 may have a bristle length of 1.2 mm and a pitch of 1 mm.

In some embodiments, the collection layer 120 with bristles 200 may form a hollow cylinder or be coupled to a hollow cylinder. The hollow cylinder may be coupled to the elongated body 110 and may form the inner cavity configured to retain the sample. The collection layer 120 with bristles 200 may include bristles 200 extending from the hollow cylinder. The hollow cylinder may include apertures configured to retain the sample. For example, the bristles 200 may collect fluid using surface tension and the fluid may be retained by passing through the apertures of the hollow cylinder. The sample may be retained within an inner cavity formed by the bristles 200 and/or the hollow cylinder. The inner diameter of the hollow cylinder may be the inner cavity. The inner cavity may be formed by the curved outer walls of the hollow cylinder from which the bristles 200 extend. Additionally, and/or alternatively, the inner cavity may extend from the outer surface formed by the bristles 200 to the elongated body 110. The apertures through which the sample passes may retain the sample until the transportation for elution. In some embodiments, the inner cavity may act as a collector and enhance exposure to the transport medium after collection. In some embodiments, the hollow cylinder may have a diameter of 1.5 mm, 2.5 mm, 3.5 mm, or 4.5 mm. The hollow cylinder having a smaller diameter may have longer bristles 200. For example, a hollow cylinder having a diameter of 1.5 mm may have bristles 200 extending 3.5 mm in length. In another example, a hollow cylinder having a diameter of 2.5 mm may have bristles 200 extending 2.5 mm in length. The bristles 200 may vary in density along the hollow cylinder to increase the sample size.

In some embodiments, the hollow cylinder may be coupled to a bulb pipette to pull in the sample from the nasal passageway and/or nasopharynx. Pulling in the sample with the bulb pipette may enable the swab 100 to function as a nasal aspiration tube in addition to collecting samples. In some embodiments, the hollow cylinder with bristles 200 may act as an extension of the handle 140 for transporting the sample from the nasal passageway and/or nasopharynx.

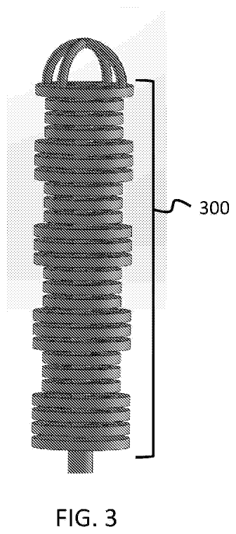

FIG. 3 depicts another example of a model illustrating a collection layer 120 having spiked disks 300. Each spiked disk 300 may form a flat, thin body that may be round or have another shape such as rectangular or prismatic. The disk(s) can vary in quantity and can be arranged in series along the length of the elongated body. The collection layer 120 having spiked disks 300 may be configured to collect a relatively large sample in comparison to previous swabs. The large sample may increase the likelihood of collecting and transporting viral nucleic acid and other pathogenic material for testing. The collection layer 120 having spiked disks 300 may also decrease discomfort in patients. The spiked disks 300 may be configured to retain a sample from a nasal passageway, the nasopharynx, and/or the like. The collection layer 120 having spiked disks 300 may facilitate elution capabilities, the performance in wet and dry nasal environments, and the decrease in the amount of material needed to manufacture the swab 100.

The collection layer 120 having spiked disks 300 may include an arrangement of solid disks of varying radii. The spiked disks 300 may be stacked on top of each other with varying radial protrusions. The collection layer 120 with spiked disks 300 may include spiked disks 300 extending from the elongated body 110. The spiked disks 300 may extend outward along an axis perpendicular (or at any angle) to the axis of the elongated body 110. The spiked disks 300 may define an outer surface of the collection layer 120. The spiked disks 300 may be configured to capture sample material from the nasal passageway, the nasopharynx, and/or the like. The spiked disks 300 may form an inner cavity extending below the outer surface of the spiked disks 300. The spiked disks 300 may collect fluid using surface tension and abrade the contact area to collect the sample. The sample may become trapped between gaps in the spiked disks 300. In some embodiments, the collection layer 120 with spiked disks 300 may be configured to form pores. The pores may be configured to retain samples from the nasal passageway and/or the nasopharynx. The pores may be configured to facilitate a capillary action to capture and retain samples from the nasal passageway and/or the nasopharynx. In some embodiments, the spiked disks 300 may have varying radii to increase abrasion and the unevenness to enhance the porosity of the spiked disks 300 and to increase the likelihood of gathering a higher sample in the pores.

The sample may be retained within the inner cavity formed by the outer surface of the spiked disks 300. The inner cavity may be configured to retain the sample. For example, the spiked disks 300 may collect fluid using surface tension and the sample may be retained within the inner cavity formed by the outer walls of the spiked disks 300. The inner cavity may be situated below an outer surface formed by the spiked disks 300 of the collection layer 120. The inner cavity may extend from the outer surface formed by the spiked disks 300 to the elongated body 110. The spiked disks 300 may include gaps through which the sample passes to retain the sample until the transportation for elution. In some embodiments, the inner cavity may act as a collector and enhance exposure to the transport medium after collection.

In some embodiments, the inner cavity formed by the spiked disks 300 may be coupled to a bulb pipette to pull in the sample from the nasal passageway and/or nasopharynx. Pulling in the sample with the bulb pipette may enable the swab 100 to function as a nasal aspiration tube in addition to collecting samples. In some embodiments, the spiked disks 300 may act as an extension of the handle 140 for transporting the sample from the nasal passageway and/or nasopharynx.

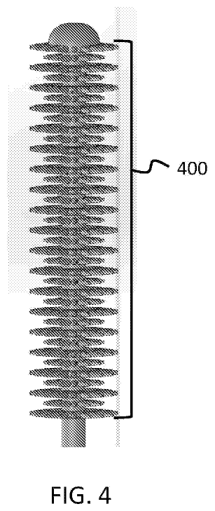

FIG. 4 depicts another example of a model illustrating a collection layer 120 having interconnecting disks 400. The collection layer 120 having interconnecting disks 400 may be configured to collect a relatively large sample in comparison to previous swabs. The large sample may increase the likelihood of collecting and transporting viral nucleic acid and other pathogenic material for testing. The collection layer 120 having interconnecting disks 400 may also decrease discomfort in patients. The interconnecting disks 400 may be configured to retain a sample from a nasal passageway, the nasopharynx, and/or the like. The collection layer 120 having interconnecting disks 400 may facilitate elution capabilities, the performance in wet and dry nasal environments, and the decrease in the amount of material needed to manufacture the swab 100.

The collection layer 120 having interconnecting disks 400 may include an arrangement of spoked circles of varying radii. The spoked circles may be stacked on top of each other with varying radial protrusions. The spoked circles may be compressible and malleable while holding the sample through surface tension. The collection layer 120 with the interconnecting disks 400 may include spoked circles extending from the elongated body 110. The interconnecting disks 400 may extend outward along an axis perpendicular (or at any angle) to the axis of the elongated body 110. The interconnecting disks 400 may define an outer surface of the collection layer 120. The interconnecting disks 400 may be configured to capture sample material from the nasal passageway, the nasopharynx, and/or the like. The interconnecting disks 400 may form an inner cavity extending below the outer surface of the interconnecting disks 400. The interconnecting disks 400 may collect fluid using surface tension and abrade the contact area to collect the sample. The sample may become trapped between gaps in the interconnecting disks 400. In some embodiments, the collection layer 120 with interconnecting disks 400 may be configured to form pores. The pores may be configured to retain samples from the nasal passageway and/or the nasopharynx. The pores may be configured to facilitate a capillary action to capture and retain samples from the nasal passageway and/or the nasopharynx. In some embodiments, the interconnecting disks 400 may have varying radii to increase abrasion and the unevenness to enhance the porosity of the interconnecting disks 400 and to increase the likelihood of gathering a higher sample in the pores. In some embodiments, the interconnecting disks 400 may be separated by a distance of 0.25 mm, 0.4 mm, 0.5 mm, 0.6 mm, 0.7 mm, 0.8 mm, 0.9 mm, and 1.0 mm.

The sample may be retained within the inner cavity formed by the outer surface of the interconnecting disks 400. The inner cavity may be configured to retain the sample. For example, the interconnecting disks 400 may collect fluid using surface tension and the sample may be retained within the inner cavity formed by the outer walls of the interconnecting disks 400. The inner cavity may be situated below an outer surface formed by the interconnecting disks 400 of the collection layer 120. The inner cavity may extend from the outer surface formed by the interconnecting disks 400 to the elongated body 110. The interconnecting disks 400 may include gaps through which the sample passes to retain the sample until the transportation for elution. In some embodiments, the inner cavity may act as a collector and enhance exposure to the transport medium after collection. In some embodiments, the interconnecting disks 400 may impart shearing stress on the sample in response to being vortexed to enhance elution.

In some embodiments, the inner cavity formed by the interconnecting disks 400 may be coupled to a bulb pipette to pull in the sample from the nasal passageway and/or nasopharynx. Pulling in the sample with the bulb pipette may enable the swab 100 to function as a nasal aspiration tube in addition to collecting samples. In some embodiments, the interconnecting disks 400 may act as an extension of the handle 140 for transporting the sample from the nasal passageway and/or nasopharynx.

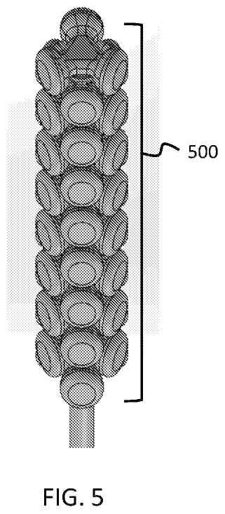

FIG. 5 depicts another example of a model illustrating a collection layer 120 having triangular disks 500. The triangular disks 500 may have a triangular profile based on three protruding members that extend from the center of the disks. The triangular profile of the triangular disks 500 may be recognizable by viewing a cross-section of the width of the elongated body 110. The collection layer 120 having triangular disks 500 may be configured to collect a relatively large sample in comparison to previous swabs. The large sample may increase the likelihood of collecting and transporting viral nucleic acid and other pathogenic material for testing. The collection layer 120 having triangular disks 500 may also decrease discomfort in patients. The triangular disks 500 may be configured to retain a sample from a nasal passageway, the nasopharynx, and/or the like. The collection layer 120 having triangular disks 500 may facilitate elution capabilities, the performance in wet and dry nasal environments, and the decrease in the amount of material needed to manufacture the swab 100.

The collection layer 120 having triangular disks 500 may include an arrangement of disks having a center portion with three protruding members extending from the center portion. The three protruding members may be equally spaced from one another. A circular protrusion may extend from a distal end of each of the protruding members. The circular protrusion may extend in a direction perpendicular (or at any angle) to the protruding members. The triangular disks 500 may be staggered relative to each other such that the circular projections fit into the cavities of adjacent triangular disks. The triangular disks 500 may extend outward along an axis perpendicular (or at any angle) to the axis of the elongated body 110. The triangular disks 500 may enable fluid to be collected and stored in a continuous channel. The triangular disks 500 may be connected to one another with a flexible central support that deforms from its own weight when held horizontally to match the incline of the bottom of the nasal cavity. The collection layer 120 with the triangular disks 500 may extend from the elongated body 110. The triangular disks 500 may define an outer surface of the collection layer 120. The triangular disks 500 may be configured to capture sample material from the nasal passageway, the nasopharynx, and/or the like. The triangular disks 500 may form an inner cavity extending below the outer surface of the triangular disks 500. The triangular disks 500 may collect fluid using surface tension and abrade the contact area to collect the sample. The sample may become trapped between gaps in the triangular disks 500. In some embodiments, the collection layer 120 with triangular disks 500 may be configured to form pores. The pores may be configured to retain samples from the nasal passageway and/or the nasopharynx. The pores may be configured to facilitate a capillary action to capture and retain samples from the nasal passageway and/or the nasopharynx. In some embodiments, the triangular disks 500 may have varying radii to increase abrasion and the unevenness to enhance the porosity of the triangular disks 500 and to increase the likelihood of gathering a higher sample in the pores.

The sample may be retained within the inner cavity formed by the outer surface of the triangular disks 500. The inner cavity may be configured to retain the sample. For example, the triangular disks 500 may collect fluid using surface tension and the sample may be retained within the inner cavity formed by the outer walls of the triangular disks 500. The inner cavity may be situated below an outer surface formed by the triangular disks 500 of the collection layer 120. The inner cavity may extend from the outer surface formed by the triangular disks 500 to the elongated body 110. The triangular disks 500 may include gaps through which the sample passes to retain the sample until the transportation for elution. In some embodiments, the inner cavity may act as a collector and enhance exposure to the transport medium after collection. In some embodiments, the triangular disks 500 may be interconnected with a tensioned string. The tensioned string may enable a clean breakaway when the tensioned string is cut or disconnected.

In some embodiments, the inner cavity formed by the triangular disks 500 may be coupled to a bulb pipette to pull in the sample from the nasal passageway and/or nasopharynx. Pulling in the sample with the bulb pipette may enable the swab 100 to function as a nasal aspiration tube in addition to collecting samples. In some embodiments, the triangular disks 500 may act as an extension of the handle 140 for transporting the sample from the nasal passageway and/or nasopharynx.

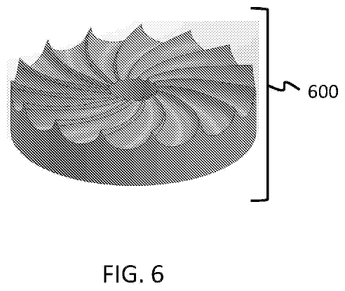

FIGS. 6-9 illustrate collection tips that may be situated at the distal end of the elongated body 110. The collection tip 130 may include a geometric layout configured to enhance the sample collected. FIGS. 6-9 illustrate various embodiments of a collection tip 130 having various geometric layouts that may include, for example, spirals 600, cylindrical protrusions 700, arches 800, and lateral-ringed arches 900, which are non-limiting examples of the geometric layouts of the collection tip 130.

FIG. 6 depicts an example of a model illustrating a collection tip 130 having spirals 600. The collection tip 130 having spirals 600 may have direct contact with the nasal cavity. The collection tip 130 having spirals 600 may be configured to collect a relatively large sample in comparison to previous swab tips. The large sample may increase the likelihood of collecting and transporting viral nucleic acid and other pathogenic material for testing. The collection tip 130 having spirals 600 may also decrease discomfort in patients. The spirals 600 may be configured to retain a sample from a nasal passageway, the nasopharynx, and/or the like. The collection tip 130 having spirals 600 may facilitate elution capabilities, the performance in wet and dry nasal environments, and the decrease in the amount of material needed to manufacture the swab 100.

The collection tip 130 having spirals 600 may include an arrangement of sloping and spiraling grooves that meet in the center. The sloping and spiraling grooves may include a tapered point for scraping the fluid and solid samples from the nasal passageway and/or the nasopharynx. The center may have a hole or well through which the sample may be retained. The collection tip 130 with the spirals 600 may be located at the distal end of the elongated body 110. The spirals 600 may define an outer tip surface of the collection tip 130. The spirals 600 may be configured to capture sample material from the nasal passageway, the nasopharynx, and/or the like. The spirals 600 may form a tip cavity extending below the outer tip surface of the spirals 600. The spirals 600 may collect fluid using surface tension and abrade the contact area to collect the sample in the tip cavity. In some embodiments, the collection tip 130 with spirals 600 may be configured to form pores between the grooves. The pores may be configured to retain samples from the nasal passageway and/or the nasopharynx. The pores may be configured to facilitate a capillary action to capture and retain samples from the nasal passageway and/or the nasopharynx.

The sample may be retained within the tip cavity formed by the outer tip surface of the spirals 600. The inner cavity may be configured to retain the sample. For example, the spirals 600 may collect fluid using surface tension and the sample may be retained within the tip cavity formed by the outer walls of the spirals 600. The tip cavity may be situated below an outer tip surface formed by the spirals 600 of the collection tip 130. The tip cavity may extend from the outer tip surface formed by the spirals 600 to the inner cavity of the collection tip 130. In some embodiments, the tip cavity may act as a collector and enhance exposure to the transport medium after collection.

The collection tip 130 may be configured to cover the inner cavity of the collection layer 120. In some embodiments, the tip cavity formed by the spirals 600 may be coupled to a bulb pipette to pull in the sample from the nasal passageway and/or nasopharynx. Pulling in the sample with the bulb pipette may enable the swab 100 to function as a nasal aspiration tube in addition to collecting samples. In some embodiments, the spirals 600 may act as an extension of the handle 140 for transporting the sample from the nasal passageway and/or nasopharynx.

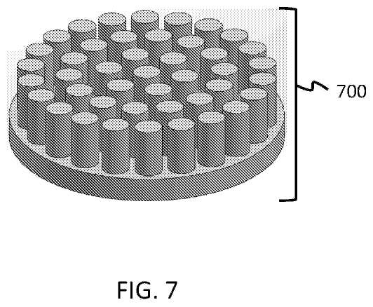

FIG. 7 depicts another example of a model illustrating a collection tip 130 having cylindrical protrusions 700. The collection tip 130 having cylindrical protrusions 700 may have direct contact with the nasal cavity. The collection tip 130 having cylindrical protrusions 700 may be configured to collect a relatively large sample in comparison to previous swab tips. The large sample may increase the likelihood of collecting and transporting viral nucleic acid and other pathogenic material for testing. The collection tip 130 having cylindrical protrusions 700 may also decrease discomfort in patients. The cylindrical protrusions 700 may be configured to retain a sample from a nasal passageway, the nasopharynx, and/or the like. The collection tip 130 having cylindrical protrusions 700 may facilitate elution capabilities, the performance in wet and dry nasal environments, and the decrease in the amount of material needed to manufacture the swab 100.

The collection tip 130 having cylindrical protrusions 700 may be mounted on a surface of a disk. Each protrusion of the cylindrical protrusions 700 may be extending members from the surface of the disk. Each protrusion of the cylindrical protrusions 700 may extend along an axis parallel to an axis of the elongated body 110. The cylindrical protrusions 700 may be spaced to allow for the collection of samples between the cylindrical protrusions 700. The cylindrical protrusions 700 may be malleable and configured to compress to maximize contact with the surface of the nasal cavity. The collection tip 130 with the cylindrical protrusions 700 may be located at the distal end of the elongated body 110. The cylindrical protrusions 700 may define an outer tip surface of the collection tip 130. The cylindrical protrusions 700 may be configured to capture sample material from the nasal passageway, the nasopharynx, and/or the like. The cylindrical protrusions 700 may form a tip cavity extending below the outer tip surface of the cylindrical protrusions 700. The cylindrical protrusions 700 may collect fluid using surface tension and abrade the contact area to collect the sample in the tip cavity. The sample may become trapped between gaps of the cylindrical protrusions 700. In some embodiments, the collection tip 130 with cylindrical protrusions 700 may be configured to form pores between the cylindrical protrusions 700. The pores may be configured to retain samples. The pores may be configured to facilitate a capillary action to capture and retain samples from the nasal passageway and/or the nasopharynx.

The sample may be retained within the tip cavity formed by the outer tip surface of the cylindrical protrusions 700. The inner cavity may be configured to retain the sample. For example, the cylindrical protrusions 700 may collect fluid using surface tension and the sample may be retained within the tip cavity formed by the outer walls of the cylindrical protrusions 700. The tip cavity may be situated below an outer tip surface formed by the cylindrical protrusions 700 of the collection tip 130. The tip cavity may extend from the outer tip surface formed by the cylindrical protrusions 700 to the inner cavity of the collection tip 130. The cylindrical protrusions 700 may include gaps through which the sample passes to retain the sample until the transportation for elution. In some embodiments, the tip cavity may act as a collector and enhance exposure to the transport medium after collection.

The collection tip 130 may be configured to cover the inner cavity of the collection layer 120. In some embodiments, the tip cavity formed by the cylindrical protrusions 700 may be coupled to a bulb pipette to pull in the sample from the nasal passageway and/or nasopharynx. Pulling in the sample with the bulb pipette may enable the swab 100 to function as a nasal aspiration tube in addition to collecting samples. In some embodiments, the cylindrical protrusions 700 may act as an extension of the handle 140 for transporting the sample from the nasal passageway and/or nasopharynx.

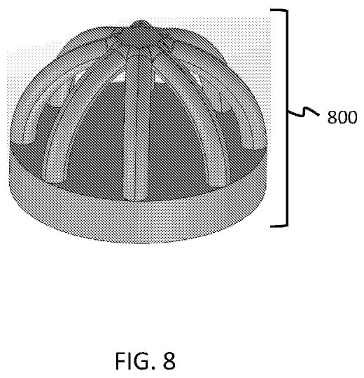

FIG. 8 depicts another example of a model illustrating a collection tip 130 having arches 800. The collection tip 130 having arches 800 may have direct contact with the nasal cavity. The collection tip 130 having arches 800 may be configured to collect a relatively large sample in comparison to previous swab tips. The large sample may increase the likelihood of collecting and transporting viral nucleic acid and other pathogenic material for testing. The collection tip 130 having arches 800 may also decrease discomfort in patients. The arches 800 may be configured to retain a sample from a nasal passageway, the nasopharynx, and/or the like. The collection tip 130 having arches 800 may facilitate elution capabilities, the performance in wet and dry nasal environments, and the decrease in the amount of material needed to manufacture the swab 100.

The collection tip 130 having arches 800 may include an arrangement of compressible arches that connect at the top to form a dome. The collection tip 130 having arches 800 may be mounted on a surface of a disk. The arches 800 may be spaced to allow for the collection of samples between the arches 800. The arches 800 may be malleable and configured to compress to maximize contact with the surface of the nasal cavity. The collection tip 130 with the arches 800 may be located at the distal end of the elongated body 110. The arches 800 may define an outer tip surface of the collection tip 130. The arches 800 may be configured to capture sample material from the nasal passageway, the nasopharynx, and/or the like. The arches 800 may form a tip cavity extending below the outer tip surface of the arches 800. The arches 800 may collect fluid using surface tension and abrade the contact area to collect the sample in the tip cavity. The sample may become trapped between gaps of the arches 800. In some embodiments, the collection tip 130 with arches 800 may be configured to form pores between the arches 800. The pores may be configured to retain samples. The pores may be configured to facilitate a capillary action to capture and retain samples from the nasal passageway and/or the nasopharynx.

The sample may be retained within the tip cavity formed by the outer tip surface of the arches 800. The inner cavity may be configured to retain the sample. For example, the arches 800 may collect fluid using surface tension and the sample may be retained within the tip cavity formed by the outer walls of the arches 800. The tip cavity may be situated below an outer tip surface formed by the arches 800 of the collection tip 130. The tip cavity may extend from the outer tip surface formed by the arches 800 to the inner cavity of the collection tip 130. The arches 800 may include gaps through which the sample passes to retain the sample until the transportation for elution. In some embodiments, the tip cavity may act as a collector and enhance exposure to the transport medium after collection.

The collection tip 130 may be configured to cover the inner cavity of the collection layer 120. In some embodiments, the tip cavity formed by the arches 800 may be coupled to a bulb pipette to pull in the sample from the nasal passageway and/or nasopharynx. Pulling in the sample with the bulb pipette may enable the swab 100 to function as a nasal aspiration tube in addition to collecting samples. In some embodiments, the arches 800 may act as an extension of the handle 140 for transporting the sample from the nasal passageway and/or nasopharynx.

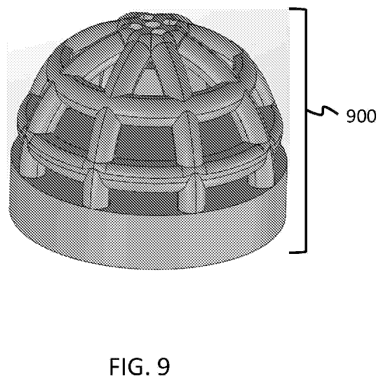

FIG. 9 depicts another example of a model illustrating a collection tip 130 having lateral-ringed arches 900. The collection tip 130 having lateral-ringed arches 900 may have direct contact with the nasal cavity. The collection tip 130 having lateral-ringed arches 900 may be configured to collect a relatively large sample in comparison to previous swab tips. The large sample may increase the likelihood of collecting and transporting viral nucleic acid and other pathogenic material for testing. The collection tip 130 having lateral-ringed arches 900 may also decrease discomfort in patients. The lateral-ringed arches 900 may be configured to retain a sample from a nasal passageway, the nasopharynx, and/or the like. The collection tip 130 having lateral-ringed arches 900 may facilitate elution capabilities, the performance in wet and dry nasal environments, and the decrease in the amount of material needed to manufacture the swab 100.

The collection tip 130 having lateral-ringed arches 900 may include an arrangement of compressible lateral-ringed arches that connect at the top to form a dome. The collection tip 130 having lateral-ringed arches 900 may be mounted on a surface of a disk. The lateral-ringed arches 900 may be spaced to allow for the collection of samples between the lateral-ringed arches 900. The lateral-ringed arches 900 may be malleable and configured to compress to maximize contact with the surface of the nasal cavity. The collection tip 130 with the lateral-ringed arches 900 may be located at the distal end of the elongated body 110. The lateral-ringed arches 900 may define an outer tip surface of the collection tip 130. The lateral-ringed arches 900 may be configured to capture sample material from the nasal passageway, the nasopharynx, and/or the like. The lateral-ringed arches 900 may form a tip cavity extending below the outer tip surface of the lateral-ringed arches 900. The lateral-ringed arches 900 may collect fluid using surface tension and abrade the contact area to collect the sample in the tip cavity. The sample may become trapped between gaps of the lateral-ringed arches 900. In some embodiments, the collection tip 130 with lateral-ringed arches 900 may be configured to form pores between the lateral-ringed arches 900. The pores may be configured to retain samples. The pores may be configured to facilitate a capillary action to capture and retain samples from the nasal passageway and/or the nasopharynx.

The sample may be retained within the tip cavity formed by the outer tip surface of the lateral-ringed arches 900. The inner cavity may be configured to retain the sample. For example, the lateral-ringed arches 900 may collect fluid using surface tension and the sample may be retained within the tip cavity formed by the outer walls of the lateral-ringed arches 900. The tip cavity may be situated below an outer tip surface formed by the lateral-ringed arches 900 of the collection tip 130. The tip cavity may extend from the outer tip surface formed by the lateral-ringed arches 900 to the inner cavity of the collection tip 130. The lateral-ringed arches 900 may include gaps through which the sample passes to retain the sample until the transportation for elution. In some embodiments, the tip cavity may act as a collector and enhance exposure to the transport medium after collection.

The collection tip 130 may be configured to cover the inner cavity of the collection layer 120. In some embodiments, the tip cavity formed by the lateral-ringed arches 900 may be coupled to a bulb pipette to pull in the sample from the nasal passageway and/or nasopharynx. Pulling in the sample with the bulb pipette may enable the swab 100 to function as a nasal aspiration tube in addition to collecting samples. In some embodiments, the lateral-ringed arches 900 may act as an extension of the handle 140 for transporting the sample from the nasal passageway and/or nasopharynx.

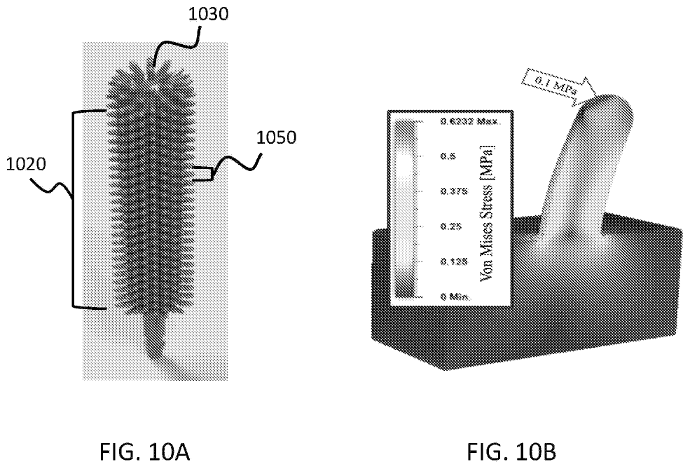

FIG. 10A depicts an example of a model illustrating a silicone collection layer and a silicone collection tip. Silicones may come in a wide range of stiffnesses. The silicone collection layer 1020 and the silicone collection tip 1030 may be made of silicone having a stiffness adapted to the nasal passageway. For example, the silicone collection layer 1020 and the silicone collection tip 1030 may have a stiffness configured to match the stiffness of the nasal mucosa. Aligning the stiffness of the silicone collection layer 1020 to the stiffness of the nasal mucosa reduces the scraping or poking of the nasal mucosa compared to conventional swabs. The silicone stiffnesses may be adapted for other soft tissues found in the nasal passageway. In some embodiments, the silicone collection layer 1020 and the silicone collection tip 1030 are made of silicone having a lower stiffness than the soft tissues found in the nasal passageway to prevent irritation and abrasion of the soft tissues.

The silicone collection layer 1020 may have cylindrical bristles 1050 configured to contact the nasal cavity. The cylindrical bristles 1050 may have a triangular pattern or tessellation. The ends of the cylindrical bristles 1050 may have a hemispherical geometry to mitigate scraping against the nasal passageway to decrease patient discomfort. The cylindrical bristles 1050 may be spaced to allow for the collection of samples between the cylindrical bristles 1050. The cylindrical bristles 1050 may be malleable and configured to compress to maximize contact with the surface of the nasal passageway. The cylindrical bristles 1050 may be configured to retain a relatively large sample from a nasal passageway, the nasopharynx, and/or the like. The larger sample may increase the likelihood of collecting and transporting viral nucleic acid and other pathogenic material for testing.

The cylindrical bristles 1050 may extend from a surface of an inner cylinder of the silicone collection layer 1020 and may extend at a perpendicular angle relative to the surface of the inner cylinder. The inner cylinder and the silicone collection layer 1020 may be located at the distal end of the elongated body 110. In some embodiments, the distal end of the elongated body 110 may have a minor diameter that measures the diameter of the inner cylinder and excludes the cylindrical bristles 1050. The minor diameter may measure at or about 3 mm. In some embodiments, the distal end of the elongated body 110 may have a major diameter that includes the cylindrical bristles 1050 and the inner cylinder. The outer diameter with the cylindrical bristles 1050 may measure at or about 5.4 mm. In some embodiments, the cylindrical bristles 1050 may have a FEM-guided bristle geometry, and a bristle diameter may have a measure at or about 0.5 mm, and the bristle length may measure at or about 0.75 mm, 1 mm, 1.2 mm, or 1.5 mm.

The silicone collection layer 1020 and the silicone collection tip 1030 may have the same geometry. In some embodiments, the silicone collection layer 1020 and the silicone collection tip 1030 may have the same triangular pattern or tessellation of the cylindrical bristles 1050. The silicone collection tip 1030 may be hemispherical and have an outer tip surface with the cylindrical bristles 1050. The cylindrical bristles 1050 may extend from the outer tip surface at an angle relative to the outer tip surface. The cylindrical bristles 1050 may form a tip cavity extending below the outer tip surface of the cylindrical bristles 1050. The sample may become trapped between gaps of the cylindrical bristles 1050. The cylindrical bristles 1050 may collect fluid using surface tension and abrade the contact area to collect the sample in the tip cavity. For example, the cylindrical bristles 1050 may collect fluid using surface tension and the sample may be retained within the tip cavity formed by the outer walls of the cylindrical bristles 1050. The tip cavity may be situated below an outer tip surface formed by the cylindrical bristles 1050 of the silicone collection tip 1030. The sample may be retained within the silicone collection tip 1030 formed by the outer tip surface and the cylindrical bristles 1050. The tip cavity may be configured to retain the sample. The tip cavity may extend from the outer tip surface formed by the cylindrical bristles 1050 to the inner cylinder of the silicone collection layer 1020. The cylindrical bristles 1050 may include gaps through which the sample passes to retain the sample until the transportation for elution.

FIG. 10B depicts an example of a bristle stress test of a silicone cylindrical bristle. The cylindrical bristles 1050 at the silicone collection layer 1020 and the silicone collection tip 1030 may have a lower stiffness compared to conventional nasopharyngeal swabs. The silicone collection layer 1020 and the silicone collection tip 1030 may be configured to absorb the impact of the swab 100 colliding with the nasopharynx. The cylindrical bristles 1050 may be mechanically modeled to measure stiffness to determine whether the cylindrical bristles 1050 are sufficiently compliant to bend when in contact with the soft tissues in the nasal passageway (e.g., nasal mucosa).

The cylindrical bristles 1050 may be sufficiently soft and compliant such that they bend rather than scape when in contact with the soft tissues in the nasal passageway (e.g., nasal mucosa). The cylindrical bristles 1050 may have a tensile strength in the range of 1-18 MPa to match the tensile strength of the orinasal tissues having 1-18 MPa. For example, the cylindrical bristles may have a tensile strength of 3 MPa for collecting a sample at the nasal mucosa having a stiffness at or about 1-10 MPa. In another example, the cylindrical bristles 1050 may have a tensile strength in the range of 11 MPa to match the tensile strength of the hard palate having a stiffness at or about 18 MPa. The cylindrical bristles 1050 may have a bending compliance of approximately 150°/MPa for bristles measuring 0.5 mm in diameter by 1.2 mm in length with the pressure loaded at the tip. In some embodiments, the bending compliance of the cylindrical bristles 1050 may be measured by applying a pressure to one side of the tip at the bristle and its resulting deflection is measured to determine the angular compliance of the bristle. In some embodiments, the testing may apply a maximum pressure and determine that the von Mises stress of the cylindrical bristles 1050 is within an elastic range. In some embodiments, the cylindrical bristles 1050 may bend well below the tensile strength of the nasal mucosa.

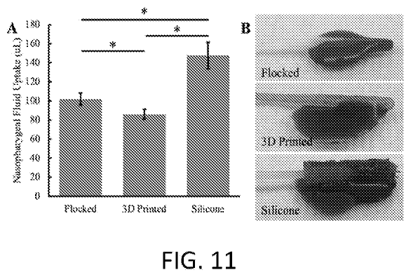

FIG. 11 depicts an example of a fluid uptake test results between swabs having a flocked collection layer, a 3-D printed collection layer, and a silicone collection layer. The silicone collection layer 1020 and silicone collection tip 1030 may exceed the performance characteristics of conventional (e.g., flocked) and novel (e.g., 3D printed) swabs. In some embodiments, the silicone swab may pick up approximately 50% more sample material than either the flocked or 3D-printed swab.

The silicone collection layer 1020 and the silicone collection tip 1030 may use a dense array of pliable silicone cylindrical bristles 1050 to wick up nasal secretions without scraping or damaging the soft tissues in the nasal passageway (e.g., nasal mucosa). Additionally, the cylindrical bristles 1050 may be configured to collect a relatively large sample in comparison to existing swab tips. The silicone collection layer 1020 having cylindrical bristles 1050 may facilitate elution capabilities, the performance in wet and dry nasal environments, and the decrease in the amount of material needed to manufacture the swab 100. In some embodiments, pre-wetting the silicone collection layer 1020 and the silicone collection tip 1030 may facilitate increased specimen update for qPCR assays. In some embodiments, the silicone collection layer 1020 and the silicone collection tip 1030 are made of silicone having a lower stiffness than other nasopharyngeal swabs.

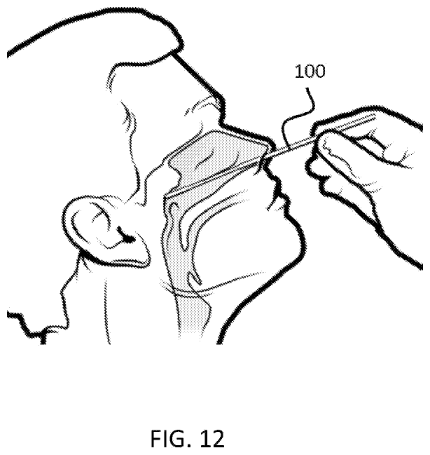

FIG. 12 depicts an example of display illustrating a swab 100 being inserted into a nasal passageway. The swab 100 may be configured to be inserted into a nasal passageway. Compared to previous swabs, the swab 100 may be configured to collect a large sample with a collection layer 120 and a collection tip 130. The large sample may also increase the likelihood of transporting viral nucleic acid (e.g., nucleic acid of COVID-19) and other pathogenic material for testing.

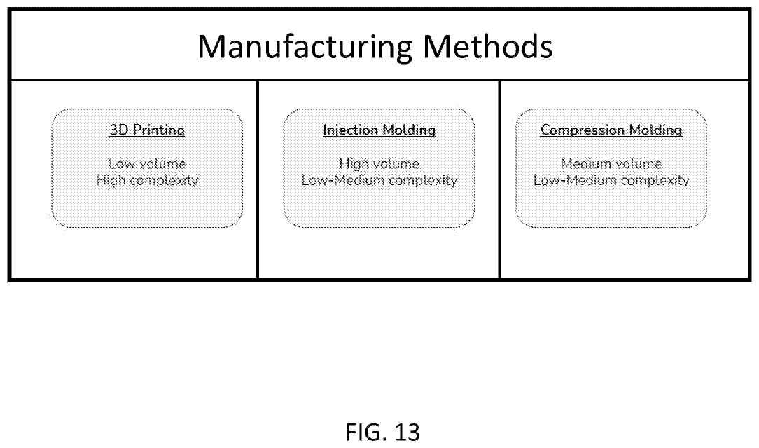

FIG. 13 depicts an example of a table illustrating the manufacturing methods of a swab 100. The swab 100 may be manufactured using 3D printing, injection molding, or compression molding. 3D printing may be advantageous for manufacturing a low volume of swabs that are structurally complex. Injection molding may be advantageous for manufacturing a high volume of swabs that are less structurally complex. Compression molding may be advantageous for manufacturing a medium volume of swabs that are less structurally complex relative to 3D printed swabs. The swab 100 may be compatible with liquid silicone rubber injection molding. The swab 100 manufactured using silicone may have more horizontal uptake than 3D-printed or flocked swabs.

In some embodiments, the swab 100 may be created by forming a collection layer 120 at a distal end of an elongated body 110, the collection layer 120 having a first geometric layout and configured to collect samples in the nasal passageway. Additionally, the swab 100 may be created by forming a collection tip 130 at the distal end of the elongated body 110, the collection tip 130 having a second geometric layout and configured to collect samples in the nasal passageway.

In some embodiments, the swab 100 may be manufactured to be a dissolvable swab. The dissolvable swab may increase the likelihood of complete elution of the sample from the swab 100. The collection layer 120 and/or the collection tip 130 may dissolve in the transport medium to increase the likelihood of complete dispersion of the sample from the swab 100. In some embodiments, the sacrificial coating may be applied to the swab 100 that acts as a barrier between the swab 100 and the sample. The sacrificial coating may be configured to be easily removed from the swab 100. The sacrificial coating may be made of poly(vinyl alcohol) or PVA.

In some embodiments, the swab 100 may be charged. The swab 100 may be created by applying a surface treatment to the collection layer 120 and the collection tip 130 where the surface treatment may be configured to positively charge the collection layer 120 and the collection tip 130. The swab 100 may be positively charged to attract negatively charged viral RNA and mucus. A surface treatment may be applied to the swab 100 to positively charge the swab 100. A material on opposite ends of the triboelectric series may be selected to produce a buildup of static charge on the collection layer 120 and the collection tip 130. Materials, such as nylon, may have a tendency to give away electrons and become positively charged. In some embodiments, nylon may lose electrons and become positively charged when rubbed with another material, especially a material that has a lower affinity for electrons than nylon. In some embodiments, an 02/plasma treatment may be applied to the swab 100 to create a hydrophilic surface. The 02/plasma treatment may include an ion beam irradiation applied to the swab 100 to create hydrophilic functional groups. For example, the ion beam irradiation may create a hydrophilic functional group including —C—O—, —(C═O)—, and —(C═O)—O—.

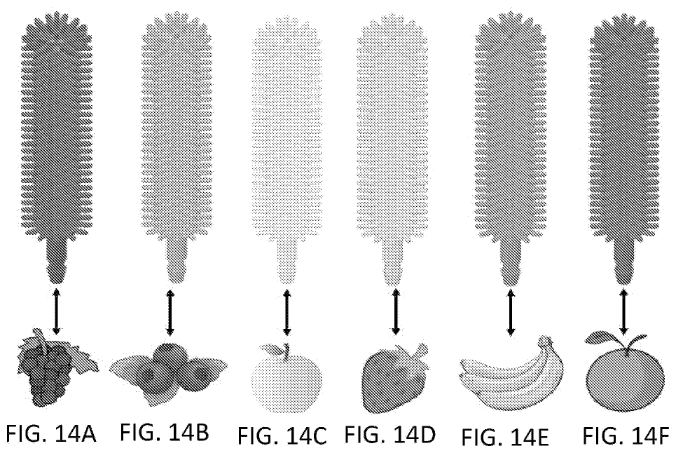

FIGS. 14A-F depict an example of a variety of colored and scented swabs. The option to choose a colored swab or a scented swab may be make the experience more enjoyable for pediatric patients by giving them control over some aspect of the procedure. For example, a colored swab may include a red swab, an orange swab, a yellow swab, a green swab, a purple swab, or a blue swab. The scented swab may be paired with a scent corresponding to the color of the swab. In some embodiments, the scented swab may be paired with a scent differing from the color of the swab. For example, the scent may correlate to the smell of bananas, blueberries, apples, strawberries, grapes, or oranges. Anosmia, or loss of smell, may be detected by subsequently asking the patient to identify the scent. Anosmia correlates highly with COVID-19 infection, so this scent test may provide immediate diagnostic value.

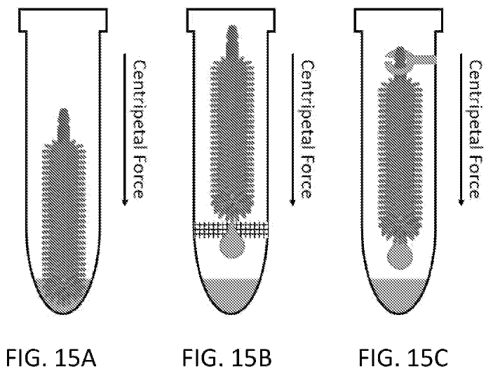

FIGS. 15A-15C depict an example of a process of centrifugal elution of a sample from a swab. As shown in FIG. 15A, the elution of a sample may occur when the swabs are submerged and mixed in viral transport media or collection fluid (e.g. 50 uL sample into 3 mL of media is a ˜60× dilution) that reduces the sensitivity of subsequent assays. Swabs may be spun in centrifuge tubes. As shown in FIG. 15B, the centrifuge tube may be fitted with a filter to extract the sample with reduced need for viral transport media or collection fluid. Silicone swabs, which do not readily wick fluid, are particularly suited to this approach as they readily de-wet. As shown in FIG. 15C, the centrifuge tube may be fitted with a clamp to hold the swab to extract the sample with reduced need for viral transport media or collection fluid. Silicone swabs, which do not readily wick fluid, are particularly suited to this approach as they readily de-wet.

In some embodiments, a coating may be applied to the swab 100. For example, a lectin coating may be applied to the swab 100 to increase the likelihood that a virus bonds to the lectin coating. The swab 100 may be formed by applying a coating to the collection layer 120 and the collection tip 130 where the coating may be configured to bond to pathogenic material located in the nasal passageway. The lectin coating may be configured to target an S protein on the surface of the virus. In some embodiments, a mannose targeting lectin coating may be applied to the swab 100.

In some embodiments, the silicone collection layer 1020 and the silicone collection tip 1030 may be manufactured from a liquid-silicone rubber injection molding. In some embodiments, a silicone swab may be formed by circularizing a planar version of the design around a cylindrical mandrel, bonding the swab, and inserting a handle. In some embodiments, a Sylgard 184 silicone may be prepared at a 10:1 ratio, degassed, casted into the molds, and cured at 100° C. for 1 hour. In some embodiments, a Sylgard 527 may be used to match the soft tissues in the nasal passageway.

In the descriptions above and in the claims, phrases such as “at least one of” or “one or more of” may occur followed by a conjunctive list of elements or features. The term “and/or” may also occur in a list of two or more elements or features. Unless otherwise implicitly or explicitly contradicted by the context in which it used, such a phrase is intended to mean any of the listed elements or features individually or any of the recited elements or features in combination with any of the other recited elements or features. For example, the phrases “at least one of A and B;” “one or more of A and B;” and “A and/or B” are each intended to mean “A alone, B alone, or A and B together.” A similar interpretation is also intended for lists including three or more items. For example, the phrases “at least one of A, B, and C;” “one or more of A, B, and C;” and “A, B, and/or C” are each intended to mean “A alone, B alone, C alone, A and B together, A and C together, B and C together, or A and B and C together.” Use of the term “based on,” above and in the claims is intended to mean, “based at least in part on,” such that an unrecited feature or element is also permissible.

The subject matter described herein can be embodied in systems, apparatus, methods, and/or articles depending on the desired configuration. The implementations set forth in the foregoing description do not represent all implementations consistent with the subject matter described herein. Instead, they are merely some examples consistent with aspects related to the described subject matter. Although a few variations have been described in detail above, other modifications or additions are possible. In particular, further features and/or variations can be provided in addition to those set forth herein. For example, the implementations described above can be directed to various combinations and subcombinations of the disclosed features and/or combinations and subcombinations of several further features disclosed above. In addition, the logic flows depicted in the accompanying figures and/or described herein do not necessarily require the particular order shown, or sequential order, to achieve desirable results. Other implementations may be within the scope of the following claims.

In the following description, for purposes of explanation, numerous specific details are set forth in order to provide a thorough understanding of the various embodiments. It should be understood that other embodiments may be utilized, and structural changes may be made without departing from the scope of the disclosed subject matter. Any combination of the following features and elements is contemplated to implement and practice the disclosure.

In the description, common or similar features may be designated by common reference numbers. As used herein, “exemplary” may indicate an example, an implementation, or an aspect, and should not be construed as limiting or as indicating a preference or a preferred implementation

The many features and advantages of the disclosure are apparent from the detailed specification, and thus, it is intended by the appended claims to cover all such features and advantages of the disclosure which fall within the true spirit and scope of the disclosure. Further, since numerous modifications and variations will readily occur to those skilled in the art, it is not desired to limit the disclosure to the exact construction and operation illustrated and described, and accordingly, all suitable modifications and equivalents may be resorted to, falling within the scope of the disclosure.

What is claimed is:

1. A swab for collecting samples in a nasal passageway, the swab comprising:
an elongated body having a distal end;
a collection layer proximate to the distal end of the elongated body, the collection layer having a first geometric layout and configured to collect samples in the nasal passageway; and
a collection tip at the distal end of the elongated body, the collection tip having a second geometric layout and configured to collect samples in the nasal passageway,
wherein the first geometric layout is different from the second geometric layout.

2. The swab of claim 1, wherein the collection layer is coupled to the collection tip at the distal end of the elongated body.

3. The swab of claim 1, wherein the first geometric layout of the collection layer forms an inner cavity configured to retain samples from the nasal passageway.

4. The swab of claim 3, wherein the inner cavity is situated below an outer surface formed by the first geometric layout of the collection layer.

5. The swab of claim 3, wherein the collection tip is configured to cover an opening of the inner cavity.

6. The swab of claim 1, wherein the collection layer and the collection tip are made of silicone, and wherein cylindrical bristles of the collection layer and the collection tip have a stiffness matching a nasal tissue stiffness.

7. The swab of claim 6, wherein the cylindrical bristles have a triangular pattern, and wherein ends of the cylindrical bristles may have a hemispherical geometry to mitigate scraping against the nasal passageway to decrease patient discomfort.

8. The swab of claim 1, wherein the collection layer is formed around a circumference of the elongated body, and wherein the first geometric layout of the collection layer forms pores configured to retain samples from the nasal passageway.

9. The swab of claim 8, wherein the pores are configured to facilitate capillary action to retain samples from the nasal passageway.

10. The swab of claim 1, wherein the elongated body is rigid and the collection layer is flexible.

11. The swab of claim 1, wherein the first geometric layout includes at least one of bristles, spiked disks, interconnecting disks, and triangular disks, and wherein the second geometric layout is at least one of spirals, cylindrical protrusions, arches, and lateral-ringed arches.

12. The swab of claim 1, further comprising:
a proximate end of the elongated body,
wherein the proximate end of the elongated body is configured to manipulate the swab for inserting the swab into the nasal passageway.

13. The swab of claim 1, wherein the elongated body is cylindrical and wherein the elongated body includes a hollow tube for transporting samples.

14. A swab system for collecting samples in a nasal passageway, the system comprising:
an elongated body having a distal end;
a collection layer configured to couple proximate to the distal end of the elongated body, the collection layer having a first geometric layout and configured to collect samples in the nasal passageway; and
a collection tip configured to couple at the distal end of the elongated body, the collection tip having a second geometric layout and configured to collect samples in the nasal passageway,
wherein the first geometric layout is different from the second geometric layout.

15. The swab system of claim 14, wherein the collection layer is configured to couple to the collection tip at the distal end of the elongated body.

16. The swab system of claim 14, wherein the first geometric layout of the collection layer is configured to form an inner cavity configured to retain samples from the nasal passageway.

17. The swab system of claim 16, wherein the collection tip is configured to cover an opening of the inner cavity.

18. A method of manufacturing a swab for collecting samples in a nasal passageway, the method comprising:
forming a collection layer at a distal end of an elongated body, the collection layer having a first geometric layout and configured to collect samples in the nasal passageway; and
forming a collection tip at the distal end of the elongated body, the collection tip having a second geometric layout and configured to collect samples in the nasal passageway,
wherein the first geometric layout is different from the second geometric layout.

19. The method of claim 18, further comprising:
applying a surface treatment to the collection layer and the collection tip, the surface treatment configured to positively charge the collection layer and the collection tip.

20. The method of claim 18, further comprising:
applying a coating to the collection layer and the collection tip, the coating configured to bond to pathogenic material located in the nasal passageway.
